(19) United States
(12) Patent Application Publication
(10) Pub. No.: US 2009/0326421 A1
(43) Pub. Date: Dec. 31, 2009

(54) Device for Producing Shock Waves

(76) Inventors: Werner Schwarze, Jena (DE); Hugo Stephan, Jena (DE)

Correspondence Address: Scott R Foster;Pandiscio & Pandiscio, 470 Totten Pond Road, Waltham, MA, 02451-1914, US

(21) Appl. No.: 12/226,253

(22) PCT Filed: May 10, 2007
(86) PCT No.: PCT/EP2007/004155

§ 371 Date: Apr. 27, 2009

(30) Foreign Application Priority Data
May 13, 2006 (DE) 10 2006 022 416.7

(51) Int. Cl. A61B 17/225

(52) U.S. Cl. 601/4

(57) ABSTRACT

The invention relates to a device (1) for producing shock waves (2), especially for medical use. Said device comprises a housing (3), being penetrable to the shock waves (2), at least one pair of electrodes (4), arranged in the interior of the housing (3), between which respective voltages for producing shock waves (2) can be adjusted, and a liquid (5), surrounding the electrodes (4) and consisting entirely or partially of a plurality of water molecules (H₂O). The aim of the invention is to substantially improve the efficiency and the service life of the device (1). For this purpose, the production of hydrogen, oxygen and hydroxide ions between the electrode lips (4) is not impeded. More specifically, the aim of the invention is to provide a device (1) which prevents hydrogen (6) from escaping, which stores it and allows its complete back reaction to water molecules.

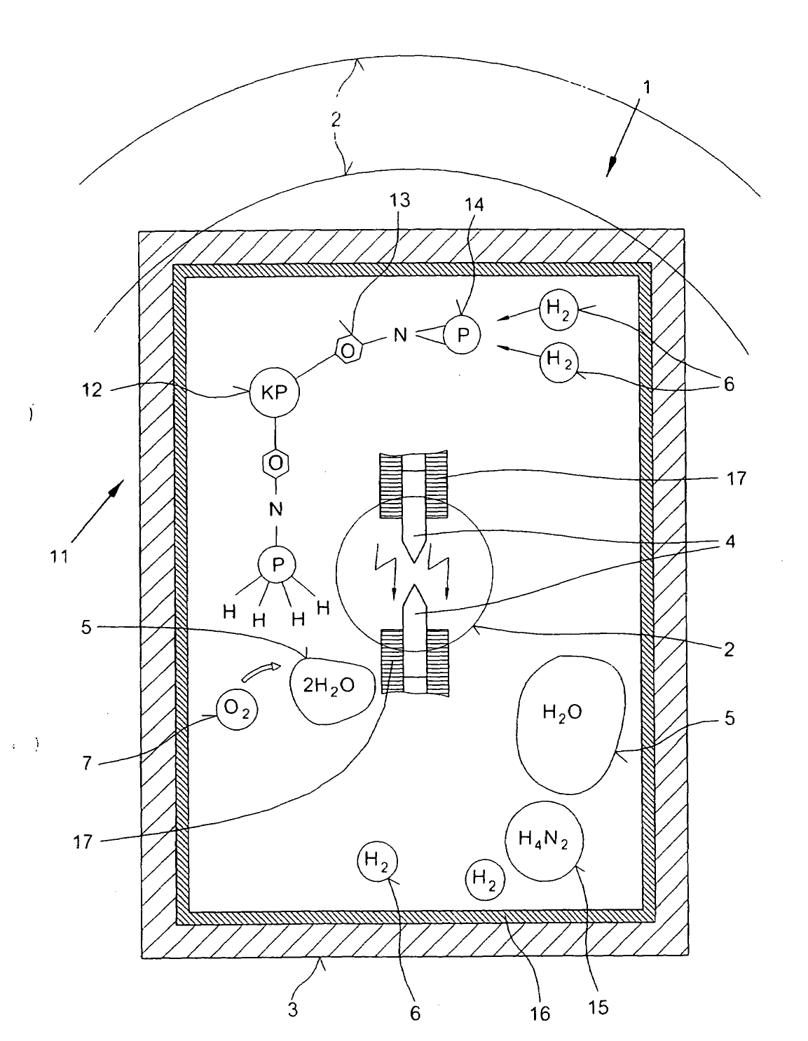

The invention relates to a device for producing shock waves, especially for medical use in accordance with the precharacterizing clause of patent claims 1 and 4.

Devices of this kind have been used for decades in medicine, for example in urology, as lithotripsers, in orthopedics for treating non-healing bone fractures or for insertion tendonitis or quite generally for promoting wound healing. In order to destroy kidney stones, for example, located in human organs from the outside of the body using acoustic shock waves, it is necessary to generate a large number of shock waves. As such, it has become apparent that the electrodes between which the shock waves are triggered by spark discharge are subject to considerable wear. Furthermore, it is necessary to accommodate the electrodes in water, since water serves as the transmission and coupling medium for the shock waves.

After the initial breakdown of the voltage applied between the electrodes tips, the discharge current oscillates back and forth between the capacitor, an inductive resistor and the electrode tips (shock circuit) several times. This results in numerous polarity reversals of the voltage at the electrode tips and a reversal of the electron flux direction between the tips. During several oscillation cycles, this leads to shock ionization of the water between the tips and in the vicinity. This shock ionization results in the partial decomposition of the water into hydrogen, oxygen and hydroxide ions. These are largely responsible for the creation of the plasma bubble between the tips, by means of which the shock wave is created and from the surface of which the shock wave is emitted.

Furthermore, it is known that the shock ionization results in the liberation of free hydrogen molecules which diffuse through the housing in which the liquid is kept within an extremely short time, thereby escaping from the area surrounding the electrode tips and consequently these molecules are no longer present in the liquid 7 surrounding the electrodes. The free oxygen molecules, in particular the dissolved gas or the gas bubbles, on the other hand, build up on the inside of the housing and impede the shock waves emitted by the electrodes.

DE 197 18 512 C1 discloses a process and a device in which it is proposed that a catalyst should be added to the liquid by means of which the electrolytic liberation of gas is entirely or partially suppressed when high voltage is applied to the electrodes and by means of which the gas liberated when the high voltage is applied to the electrodes and during electrical breakdown is entirely or partially restored to its initial condition by a catalytic effect.

This state-of-the-art technology does however suffer from the disadvantage that the described and intended suppression of gas formation when the high voltage is applied and during subsequent oscillation of the oscillating circuit impedes the expansion of the plasma bubble between the electrode tips. This reduces the efficiency of energy conversion from electrical into acoustic energy in the overall system.

Furthermore, it must be seen as disadvantageous that the reconversion of the resulting gas (H₂ and O₂) into water (H₂O) is only partially successful because hydrogen continuously escapes from the volume in the vicinity of the tips by means of diffusion, and therefore there is no reaction partner available for the oxygen.

The purpose of the present invention is therefore to provide a device of the kind mentioned in the introduction, by means of which the efficiency and service life of the device is considerably increased in comparison to the state-of-the-art, without the formation of hydrogen, oxygen and hydroxide ions between the electrode tips being prevented. Rather, it is the objective of the present invention to create a device that prevents the hydrogen from escaping, stores it and allows the back reaction into water molecules to occur in full.

This purpose is achieved by the features that are listed in the precharacterizing clause of patent claims 1 and 4. Plasma bubble creation and expansion is prevented by adding a hydrogen buffer to the liquid medium. Adding substances that liberate hydrogen molecules also means that the effectiveness of the system is maintained at an optimum level as well as ensuring that the resulting gases are available in a favorable stochiometric concentration, thereby permitting the back reaction into water to occur in full.

The hydrogen buffer can be added to the water not only in dissolved or suspended form but also as a colloidal flotation. This is even possible as a sediment because it can be swirled back up into suspension in the reaction chamber by shaking the volume or by the turbulences in the liquid generated by the shock wave itself.

Another possible way of binding the free hydrogen molecules involves applying the hydrogen buffer to the surface of the housing that surrounds the electrodes. Also, carrier materials such as fine net-like structures can contain the hydrogen buffer on their surface in the vicinity of the electrodes.

The hydrogen buffer can consist of bound or non-bound nano or microparticles, of films or sponge-like layers on surfaces. These various hydrogen buffers share the characteristic that the hydrogen atom cores liberated due to the shock ionization and which react to form hydrogen molecules are held in the liquid and that therefore the free oxygen molecules present in the liquid react with the hydrogen held fast in the hydrogen buffer thereby creating water molecules.

The drawing shows a sample embodiment configured in accordance with the present invention, the details of which are explained below. In the drawing, the only FIGURE shows a device for generating shock waves with a pair of electrodes in a housing containing water and a buffer medium.

A device 1 should enable shock waves 2 to be generated that can be used for a medical application, for example lithotripsy of kidney stones. The device 1 consists of a housing 3 that is permeable to sound within which a pair of electrodes 4 is arranged with the individual electrodes being located opposite one another. Water 5 is filled inside the housing 3. A high voltage is applied across the opposite electrodes 4, which upon its breakdown creates a plasma bubble which in turn generates a shock wave 2 in water. The shock waves 2 pass through the housing 3 to the outside and are directed by a focusing device (not illustrated) in such a way as to enable a kidney stone, for example, in the human body to be fragmented.

Due to the voltages arising between the electrodes 4, shock ionization of the flowing electrons when the voltage breakdown occurs turns water molecules into freely mobile hydrogen molecules (H₂) 6 and oxygen molecules (O₂) 7.

In order to bind the hydrogen molecules 6 in the water 5 and prevent them from escaping into the surroundings through the housing 3, there is a plurality of hydrogen buffers 11 present in the water 5. Furthermore, the inside of the housing 3 can be covered with a buffer layer 16 in order to keep the hydrogen molecules 6 inside the housing 3 and therefore in the water 5.

The buffer medium 11 can consists of, for example, synthetic resin pearls 12 with their surface consisting of nano or microparticles. A nitrogen bond 13 is provided on these surface structures, with a palladium particle 14 attached to the free end of each bond. The palladium particles 14 have a metallic outer layer by means of which the hydrogen molecules 6 present in the water 5 are bound. This therefore results in three chemical reactions between the metalized outer layer of the palladium particles 14 and the hydrogen molecules 6 present in the water:

H₂+Pd⁰→Pd⁰+2H

As soon as the oxygen molecules 7 present in the water 5 flow past the hydrogen molecules 6 bonded to the palladium particles 14, a further chemical reaction takes place, namely:

2H+O—O→2HO—OH

and this compound reacts further according to the following chemical formula

2H+2HO—OH→2H₂O.

This reaction sequence means that water 5 has been reformed from the hydrogen and oxygen molecules 6 and 7 respectively that were liberated by the electrolysis.

The buffer layer 16 that can be embodied as a film, sponge, mesh or net, can also consist of different layers, on the surface of which a hydrogen buffer such as palladium particles 14 adheres or is bound.

Furthermore, the outer surfaces of the electrodes 4 and/or the material of the electrode insulation 17 can be covered with the buffer layer 16. It is particularly advantageous if the barrier layer 16 is arranged directly in the area of the spark gap formed between the electrodes 4, because this means the oxygen molecules 7 react with the hydrogen molecules 6 in the immediate vicinity of the spark formation and are therefore created outside the plasma bubble.

Due to the chemical characteristic that hydrogen molecules 6 are extremely difficult to store and keep within the envelope, there is provision for also adding chemical substances to the water 5 by means of which additional hydrogen molecules 6 are provided. For example, hydrazine molecules 15 or dilute organic or inorganic acids or sales can be provided which liberate hydrogen 6 in the form of hydronium ions into the water 5. This is necessary in order to guarantee that the oxygen molecules 7 created by the shock ionization will always find sufficient reaction partners available for forming water molecules 5.

What is claimed is:

1 A device for producing shock waves for medical use, the device comprising a housing (3) penetrable by shock waves, at least one pair of electrodes (4) arranged in an interior of the housing (3), between which electrodes respective voltages for producing the shock waves can be adjusted, and a liquid (5) surrounding the electrodes (4) at least in part comprising a plurality of water molecules (H₂O), wherein a barrier layer (16) is provided on at least one of the outside and inside of the housing (3), said layer (16) being configured so as to bind H₂ molecules present and freely mobile in the liquid (5), and permit the shock waves to pass therethrough unimpeded.

2. The device in accordance with claim 1, wherein said barrier layer (16) is formed as a selected one of a film, sponge, mesh, and net.

3. The device in accordance with claim 1, wherein the barrier layer (16) consists of a carrier layer and an outer palladium layer.

4. A device for producing shock waves, for medical use, the device comprising a housing (3) penetrable by shock waves, at least one pair of electrodes (4) arranged in an interior of the housing (3), between which electrodes respective voltages for producing shock waves can be adjusted, and a liquid (5) surrounding the electrodes (4) and comprising at least in part a plurality of water molecules (H₂O), wherein a buffer medium (11) that absorbs hydrogen molecules (6) liberated within the liquid (5) is provided in the liquid (5), the buffer medium (11) having a surface consisting of nano and micro particles by means of which the hydrogen molecules (6) in the liquid (5) are bound, and free oxygen molecules (7) in the liquid (5) react with the hydrogen molecules (6) attached to the particles forming water molecules (5).

5. The device in accordance with claim 4, wherein the buffer medium (11) can be filled into the liquid (5) from outside the housing.

6. The device in accordance with claim 4, wherein the buffer medium comprises a plurality of synthetic resin pearls (12) having a plurality of palladium particles (14) arranged thereon by means of nitrogen bonds (13).

7. The device in accordance with claim 6, wherein the palladium particles (14) have a metallic outer layer by means of which the hydrogen molecules (6) free to move in the liquid (5) are bound.

8. The device in accordance with claim 1, wherein a selected one of hydrazine, diluted acids, and salts, are added to the liquid (5), from which the hydrogen molecules (6) can be liberated.

9. The device in accordance with claim 1, wherein surfaces of the electrodes (4) covered by the liquid (5) are at least in part covered by the barrier layer (16).
